(19) United States
(12) Patent Application Publication
(10) Pub. No.: US 2017/0083803 A1
(43) Pub. Date: Mar. 23, 2017

(54) NON-INTERNET BASED SCANNABLE AND DYNAMIC MEDICAL INFORMATION RECORD AND METHOD

(71) Applicant: James B. Broselow, Hickory, NC (US)

(72) Inventor: James B. Broselow, Hickory, NC (US)

(21) Appl. No.: 14/862,737

(22) Filed: Sep. 23, 2015

(51) Int. Cl. G06K 19/06; A61N 1/39; G06K 19/077

(52) U.S. Cl. CPC G06K 19/06037; G06K 19/0776; A61N 1/3925

(57) ABSTRACT

A method of providing medical information specific to a patient that includes determining medical information specific to a patient and encoding the medical information as a bar code image. The bar code image is applied to a patient-specific substrate. The bar code image is decoded when desired. The steps are performed without utilizing an internet connection. A related medical information card is also disclosed.

### TECHNICAL FIELD AND BACKGROUND OF THE INVENTION

This application relates to a medical information record system that is scannable and dynamic, but which is specifically intended not to be internet-based. In other words, the information is stored in a manner whereby the information stored on a physical object is not transmitted via wire or wireless means for storage beyond the physical object. The information is stored on a physical object such as a wallet-sized card on a bar code such as the Aztec-type two-dimensional printed tag with matrix symbology that can be read with bar code and other optical readers. The Aztec standard is found at ANSI/AIM BC13 ITS/97/002. A wide variety of bar code fonts and encoders are available.

The system according to the invention provides the ability to transmit in seconds significant and potentially lifesaving amounts of critical medical information offline in digital format to any computer or mobile device having the ability to read 2D bar code.

Medical records were once exclusively paper-based, with each patient having a paper “file” maintained by a physician, hospital, clinic or the like. Clearly, maintaining a paper record in a manner whereby each of several medical providers in different locations have current, consistent information was and remains a serious problem, particularly when a patient is in an emergency situation with care being administered by personnel unfamiliar with the patient. Wallet size printed cards sometimes provided to patients typically have little more than blood type and allergy information.

For this reason, much effort and money has been expended on various types of medical record storage systems, much of which is directed towards a single electronic record system that stores records of hundreds or thousands of patients that are accessible by enterprise computer-based systems from numerous locations. This type of record keeping creates its own problems, including identifying and controlling who has access to the records, who can alter the content of the records, and how to make the records available outside the home system. Ordinarily, universal access to medical records is not possible because of incompatible computer languages, programs and systems, among other reasons. Even if a computer is available, problems such as lack of use training, lack of internet access and the need for rapid treatment typically render otherwise robust systems useless in many instances.

Many such systems end up being so complicated that use of the system is minimal, resulting in fragmentary or outdated data.

### SUMMARY OF THE INVENTION

Therefore, is it an object of the invention to provide a system that permits access to important emergency information without needing to be dependent on internet or other electronic connectivity.

It is a further object of the invention to provide a digital information storage system that permits easy access, legibility and the ability to quickly modify the content of the information.

It is a further object of the invention to provide a digital information storage system that is portable and that can be updated in real time.

It is a further object of the invention to provide a digital information storage system that works well in emergency settings with no need for patient identifiers.

It is a further object of the invention to provide a digital information storage system that is modular, and therefore in which the sources of medication information can be provided from multiple sources—physicians, nurses, pharmacies, hospitals, offices, nursing homes, advocacy organizations and the like.

It is a further object of the invention to provide a digital information storage system that is easy to access and use, inexpensive, expandable and adaptable to new technology.

These and other objects and advantages of the invention are achieved by providing a method of providing medical information specific to a patient that includes determining medical information specific to a patient and encoding the medical information as a bar code image. The bar code image is applied to a patient-specific substrate. The bar code image is decoded when desired. The encoding step may optionally be performed without utilizing an internet connection. The decoding step is performed without the use of an internet connection.

According to one embodiment of, the invention, the bar code image is applied to an adhesive sticker and the patient-specific substrate comprises a card.

According to another embodiment of the invention, the medical information is encoded by entering text on a digital input device in which resides software that converts text into the bar code image.

According to another embodiment of the invention, the information is encoded by voice recognition software residing in a digital input device.

According to another embodiment of the invention, the bar code image is an Aztec bar code image.

According to another embodiment of the invention, the method includes the steps of updating the medical information, encoding the updated medical information as an updated bar code image, applying the updated bar code image to a patient-specific substrate, and when desired, decoding the updated bar code image.

According to another embodiment of the invention, the method includes the step of removing the bar code image before the updated bar code image is applied to the patient-specific substrate.

According to another embodiment of the invention, the updated bar code image is applied to an adhesive sticker and the patient-specific substrate comprises a card.

According to another embodiment of the invention, the card is plastic and the adhesive sticker is adapted to be removable from the card when desired.

According to another embodiment of the invention, a medical information record is provided that includes a patient-specific substrate and a bar code image created by determining medical information specific to a patient, encoding the medical information as a bar code image and applying the bar code image to the patient-specific substrate for subsequent decoding when desired, wherein the record is created without utilizing an interne connection.

According to another embodiment of the invention, the bar code image is printed onto an adhesive sticker and the patient-specific substrate comprises a card.

According to another embodiment of the invention, the bar code image is an Aztec bar code image.

According to another embodiment of the invention, the adhesive sticker is removable from the card for replacing on the card the adhesive sticker with an adhesive sticker having an updated bar code image.

According to another embodiment of the invention, the card includes a first adhesive sticker bearing encoded medication information and a second adhesive sticker bearing emergency medical information.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is an example of a card form of a digital information storage system according to one embodiment of the invention;

FIG. 2 is an environmental view showing one method of accessing the card form of the digital storage information system of FIG. 1; and

FIG. 3 is a screen shot displaying information of the type typically capable of being placed on such a card.

### DESCRIPTION OF THE PREFERRED EMBODIMENT AND BEST MODE

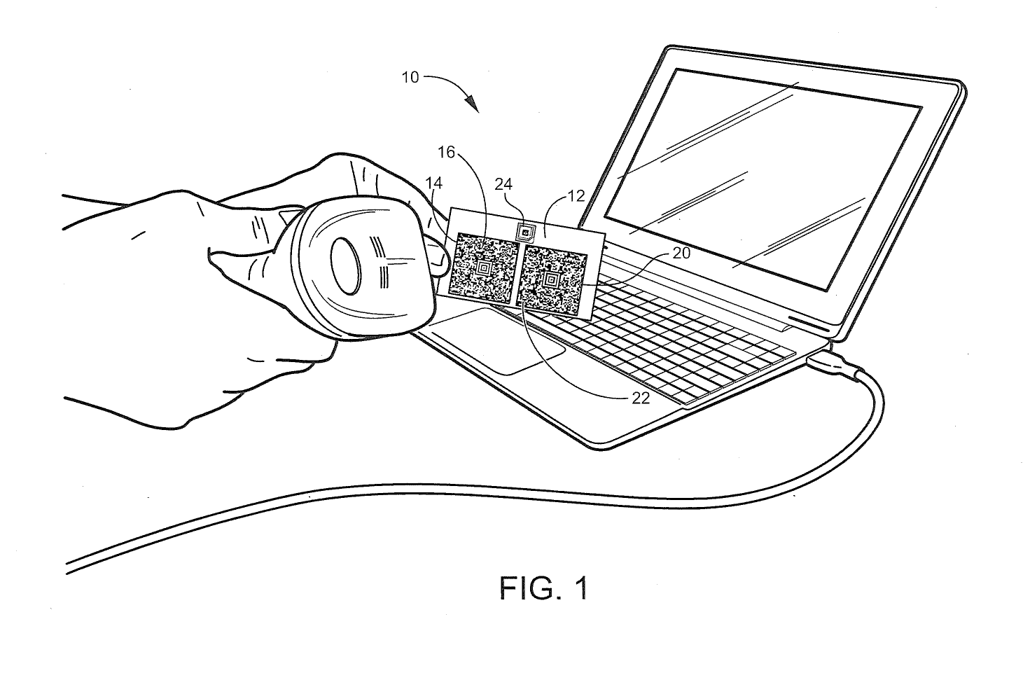

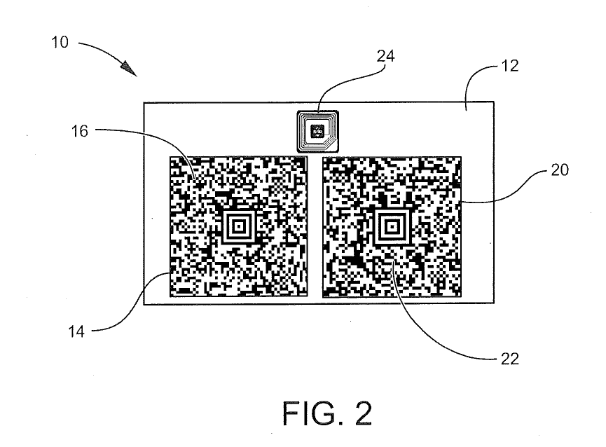

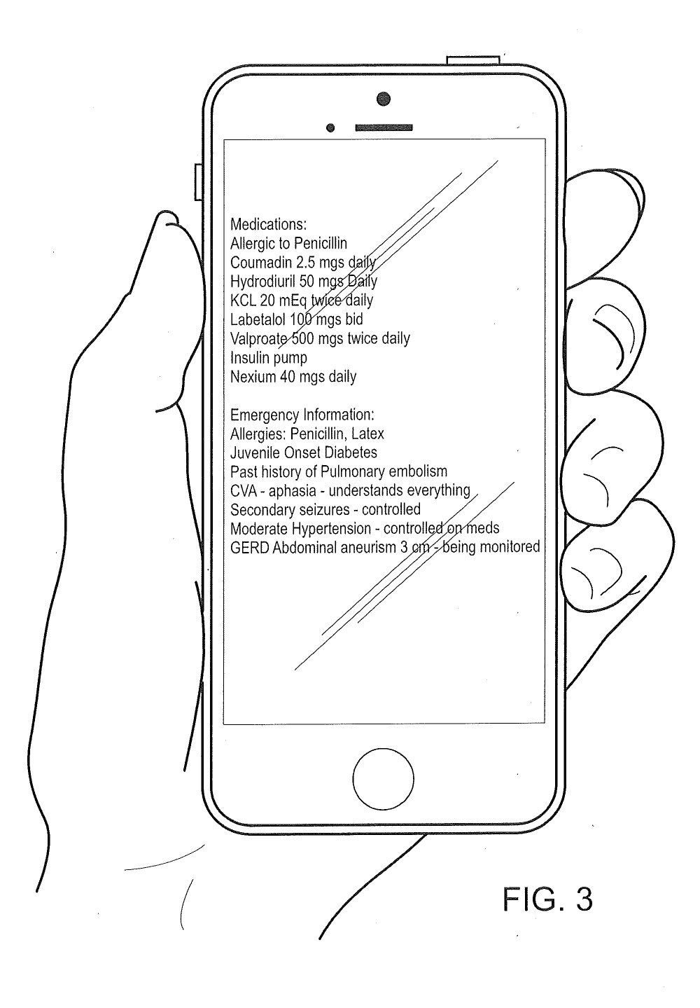

Referring now to FIGS. 1 and 2 of the drawings, a digital information storage system 10 is shown that includes a wallet card 12 on which are adhered stickers 14 and 22 on which are printed a bar code, such as Aztec-type bar code images 16 and 22. If desired, only a single bar code sticker may be utilized. However, it is preferable that the two stickers 14, 22 with the bar code images 16, 22 be utilized, one containing patient medication information, and the other containing patient information useful in a medical emergency. The card 12 is preferably plastic with a finish onto which a sticker can be adhered, but removed when desired. See FIG. 3.

An Aztec-type bar code such as images 16, 22 uses two-dimensional digital matrix symbology readable with a bar code and other optical reader. A finder structure includes a center square and a plurality of nested squares having centers that approximately coincide with the center square. A symbol descriptor located adjacent to the finder structure encodes data indicating the size of the symbol and the length of the encoded message. A reference grid extends throughout the symbol to facilitate the location of individual data elements. The data structure of the symbol is filled with data blocks which are organized into layers which surround the finder structure. Data encoded in the data blocks may be read sequentially within layers and between layers.

Technical details of the Aztec bar code are found at http://www.iso.org/iso/iso_catalogue/catalogue_tc/catalogue_detail.htm?csnumber=41548.

This coding structure is the subject of U.S. Pat. No. 5,591,956 entitled “Two Dimensional Data Encoding Structure and Symbology for Use with Optical Readers.” The entire term of the patent was dedicated to the public, as published in the Official Gazette of the United States Patent and Trademark Office, dated Jun. 17, 1997. The Aztec bar code is particularly suited to the present invention because it is compact, read by many types of readers, including smart phones and tablets, and will hold up to about 500 words of data.

Optionally, a RFID chip 24 can be placed on the card 12 so that the card 12 can be easily located, even without the intervention of the possessor. The RFID chip 24 allows for the wireless use of electromagnetic fields to transfer data for automatically identifying and tracking tags attached to objects.

The typical amount of information necessary to provide treatment, especially emergency treatment, is relatively compact. For example, the information set out below and in FIG. 3 would ordinarily be sufficient to inform emergency medical personnel about a patient's health situation:

Medications (Sticker 14):
Allergic to Penicillin
Coumadin 2.5 mgs daily
Hydrodiuril 50 mgs daily
KCL 20 mEq twice daily
Labetalol 100 mgs bid
Valproate 500 mgs twice daily
Insulin pump
Nexium 40 mgs daily

Emergency Information (Sticker 20):
Allergies: Penicillin, Latex
Juvenile Onset Diabetes
Past history of Pulmonary embolism
CVA—aphasia—understands everything
Secondary seizures—controlled
Moderate Hypertension—controlled on meds
GERD
Abdominal aneurism 3 cm—being, monitored

Bar codes 16 and 22 demonstrate that this amount of information can easily be stored on an Aztec bar code sticker as digital data. As shown in FIG. 1, medications information can be stored on the sticker 14 and emergency information can be stored on the sticker 20. The data in bar code format to be applied to stickers 14 and 20 can be quickly and easily entered by computer or other data entry device into an encoding program that converts the data into the bar code image. A conventional label printer can then print the stickers 14 and 20 for application to the card 12 or other substrate surface. Of course, without reading and decoding the bar code stickers 14 and/or 20, the data is unintelligible, preserving privacy. Because virtually any smart phone, tablet or laptop with a card reader can instantly decode the data for use, access is almost always available when needed without regard to the availability of internet or Wifi.

The system is therefore accessible in multiple situations and with minimal cost associated with accessing the content. This solution is therefore a hybrid of a paper and electronic system. These codes can be applied as single or multiple stickers that can also be fixed to a larger card or used individually, for example, on the back of a driver's license, or onto any other substrate. The stickers can be colored coded to indicate, for instance, drugs, emergency information, genomics, hospital discharge, and the like.

A significant feature is the dynamic nature of the system operation. Because the bar code can be generated and printed so quickly and inexpensively, a new bar code sticker can be printed at a doctor's office, pharmacy, hospital, emergency room, ambulance or any other location where a smart phone, tablet or laptop can transmit the data by wire or wirelessly to an inexpensive label printer. The sticker on the card can be removed and the updated sticker placed on the card. Voice recognition software is now so advanced that a keyboard input is no longer necessary. The required spoken text can be translated by software into the Aztec or other bar code image.

This system has particular application in military combat, fire fighting and similar situations. A medic may treat a patient with multiple injuries requiring tourniquets to extremities, a chest tube, dressing for open fractures, transfusions of plasma and large amounts of fluids, morphine for pain, and the like. All of this information can be typed or dictated, turned into one or multiple Aztec codes that are then affixed to a chart or even directly onto garments or equipment being worn by the patient. A smart phone and small label printer is the only hardware needed to support this critical mission.

For domestic use, the code can include the fact that a patient was started on chemotherapy and will be at risk for infections during the following two weeks. Preferred antibiotics to use, contact information for the oncologist on call, and the like. If needed for privacy, the text in the bar codes can be encrypted so that decoding is restricted to certain scanners, licensees, or a combination thereof. However, the unintelligible nature of the bar code itself preferably provides the necessary security, so that even the patient's name need not be on the card. Possession of the card or sticker together with the actual information on the card provides the necessary association between the bar code information and the patient.

Other information, for example, a living will, contact information for next of kin and the like can also be entered as bar code data. An important factor is the dynamic, easily and quickly changeable nature of the system without the need for an internet connection. Many data storage systems emphasize the need to centrally store vast amounts of data for many patients in a single location. This is important when the data must be accessed and, for example, analyzed to detect data trends across a patient population. That is not the purpose of this system. Rather, this system focuses on the immediate medical or other needs of a single patient. The absence of the requirement of an internet connection is an advantage of the system, whereas in other systems an internet connection has become a requirement of the system. Moreover, since the information is not as susceptible to hacking or other improper interception and misuse, both enhanced security and privacy is provided. This is a significant benefit, since emergency information on the bar code can be needed anywhere at any time, whether or not an internet connection is available.

A medical information record system and product is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

1. A method of providing medical information specific to a patient, comprising:
(a) determining medical information comprising a plurality of separate medical information items specific to a patient including medication and medical treatment instructions and;
(b) encoding the plurality of separate medical information items as a bar code image:
(c) applying the bar code image to a substrate associated with a specific patient; and
(d) when desired, decoding the bar code image, wherein at least step (d) is performed without utilizing an internet connection.

2. A method according to claim 1, wherein the bar code image is applied to an adhesive sticker and the patient-specific substrate comprises a card.

3. A method according to claim 1, wherein the medical information is encoded via an internet connection by entering text on a digital input device that converts text into the bar code image.

4. A method according to claim 1, wherein the information is encoded by voice recognition software residing in a digital input device.

5. A method according to claim 1, wherein the bar code image is an Aztec bar code image.

6. A method according to claim 1, and including the steps of:
(a) updating the medical information,
(b) encoding the updated medical information as an updated bar code image:
(c) applying the updated bar code image to the patient-specific substrate; and
(d) when desired, decoding the updated bar code image.

7. A method according to claim 6 and including the step of removing the bar code image before the updated bar code image is applied to the patient-specific substrate.

8. A method according to claim 7, wherein the updated bar code image is applied to an adhesive sticker and the patient-specific substrate comprises a card.

9. A method according to claim 2, wherein the card is plastic and the adhesive sticker is adapted to be removable from the card when desired.

10. (canceled)

11. A medical information record, comprising a bar code image created by determining a plurality of separate medical information items specific to a patient including medication, emergency contact information, medical treatment instructions, encoding the plurality of medical information items as a bar code image and applying the bar code image to the patient-specific substrate for subsequent decoding when desired, wherein the record is created without utilizing an internet connection.

12. A medical record according to claim 11, wherein the bar code image is printed onto an adhesive sticker and the patient-specific substrate comprises a card.

13. A medical record according to claim 11, wherein the bar code image is an Aztec bar code image.

14. A medical record according to claim 12, wherein the adhesive sticker is removable from the card for replacing on the card the adhesive sticker with an adhesive sticker having an updated bar code image.

15. A medical record according to claim 12, wherein the card includes a first adhesive sticker bearing encoded medication information and a second adhesive sticker bearing emergency medical information.
